(19) United States
(12) Patent Application Publication
(10) Pub. No.: US 2013/0288329 A1
(43) Pub. Date: Oct. 31, 2013

(54) Producing Algae Biomass Having Reduced Concentration Of Contaminants

(71) Applicants: Jose Sanchez, Los Angeles, CA (US); Nicholas Eckelberry, Los Angeles, CA (US)

(72) Inventors: Jose Sanchez, Los Angeles, CA (US); Nicholas Eckelberry, Los Angeles, CA (US)

(21) Appl. No.: 13/872,044

(22) Filed: Apr. 26, 2013

Related U.S. Application Data
(63) Continuation-in-part of application No. 13/865,087, filed on Apr. 17, 2013.
Application No. 13/865,087 is a continuation-in-part of application No. 13/753,484, filed on Jan. 29, 2013.
(60) Provisional application No. 61/592,522, filed on Jan. 30, 2012.
(60) Provisional application No. 61/625,463, filed on Apr. 17, 2012.
(60) Provisional application No. 61/649,083, filed on May 18, 2012.

(51) Int. Cl. C12N 13/00

(52) U.S. Cl. CPC C12N 13/00
USPC 435/173.9; 210/202

(57) ABSTRACT

The present invention is generally directed to a system for producing an algae biomass and wastewater that have reduced concentrations of contaminants. The algae and wastewater treated by the system of the present invention can be combined in a heterotrophic growth system in which the growth of the algae is increased due to the reduced concentration of contaminants. The algae grown in this manner also has a longer shelf life due to the lack of contaminants within the harvested algae.

### CROSS-REFERENCE TO RELATED APPLICATIONS

This application is a continuation-in-part of U.S. patent application Ser. No. 13/865,087 filed Apr. 17, 2013, titled Harvesting And Dewatering Algae Using A Two-Stage Process, which is a continuation-in-part of U.S. patent application Ser. No. 13/753,484, filed Jan. 29, 2013, titled Systems And Methods For Harvesting And Dewatering Algae, which claims priority to U.S. Provisional Patent Application No. 61/592,522, filed Jan. 30, 2012, titled Systems And Methods For Harvesting And Dewatering Algae.

U.S. patent application Ser. No. 13/865,087 also claims priority to U.S. Provisional Patent Application No. 61/625,463, filed Apr. 17, 2012, titled Solute Extraction From An Aqueous Medium Using A Modular Device, and to U.S. Provisional Patent Application No. 61/649,083, filed May 28, 2012, titled Modular Systems And Methods For Extracting A Contaminant From A Solution.

### BACKGROUND

There is a history of separating materials from liquid suspension in several industries, including the wastewater treatment industry and algae farming industry. Processes involved in achieving separation can vary, along with the desired end result. For example, in the wastewater treatment industry, the desired result is typically treated water that can be released into the environment. In contrast, in the algae farming industry, the primary desired result may be the harvest of a usable biomass for energy production.

There is a long history of electro-flocculation in the wastewater industry. It has been found to be an effective method of separating solids from fluids in the secondary stage of remediation. This waste stream contains organic material of all types and algae are considered a nuisance generated by the high nitrate count common in the stream. Therefore, efforts in algae eradication usually does not include preservation of the integrity of the mass for further uses such as pharmaceutical or other high value feedstock.

In electro-flocculation, as commonly used in wastewater treatment, a metal ion or cation is added to improve flocculation by increasing conductivity of the matrix. The following cations have lower electrode potential than H+ and are therefore considered suitable for use as electrolyte cations in these processes: Li+, Rb+, K+, Cs+, Ba2+, Sr2+, Ca2+, Na+ and Mg2+ (sodium and lithium are frequently used as they form inexpensive salts). Other metals are used in conjunction with electro-flocculation to assist in precipitation of solids from the waste water, such as iron oxides and other oxidants. These metals are extremely effective at precipitating solids out of solution; however, they taint the product and the water itself with an inorganic chemical that then must be removed or otherwise processed in the tertiary waste treatment phase.

In practice, the current used by the wastewater systems for electro-flocculation is generally low, typically under 1 amp, as the processes are carried out in large ponds and/or in conjunction with massive fluid flows typical to a waste treatment plant which can be in the millions of gallons per day. Due to the sheer size of the plants and Ohms law (I=V/R) the current requirement and the scale of the process, it is not practical to utilize high-energy electro-flocculation systems for extended periods of time. Furthermore, the deterioration and scaling of electrolytic plates operating at high current for extended periods of time precludes the effective use of this technology at high amperage. The conductivity of the waste flow therefore must be enhanced by metal ions as discussed above to lower the energy requirement and make the process practical.

In algae product farming and harvesting, the considerations are reversed as the biomass in suspension is considered an asset whose qualities must be preserved, and the use of metals taints the product irreversibly. Most methods used to dewater the algae in suspension therefore consist of centrifuge, membrane filtration, air drying with possible chemical processing and decontamination.

One approach used to dewater algae is known as Dissolved Air Flotation (DAF). Typically, this flocculation method involves the use of coagulants, emulsifiers or other chemicals in tandem with a curtain of air generated from pumps or cyclones. While this method is generally more effective from an energy standpoint than centrifuge techniques, it has the inherent drawback of requiring both chemicals and an independent tank. Furthermore, the effectiveness of the DAF system as a continuous system is hampered by the creation of bubbles as a source of turbulence within the reactor. The solution to this problem has been to increase the size of the flotation which leads to larger and larger footprints.

Additionally, the use of chemicals in dewatering commonly prevents or limits reuse of the growth water. The related prior application Ser. No. 13/274,094, filed Oct. 14, 2011 and titled Systems, Methods, and Apparatuses for Dewatering, Flocculating, and Harvesting Algae Cells, which is incorporated by reference, discloses some forms of electro-magnetic flocculation systems. That application focuses on cell lysing as the end product.

The harvesting of microorganisms and intracellular products of microorganisms such as algae shows promise as a partial or full substitute for fossil oil derivatives or other chemicals used in manufacturing products such as pharmaceuticals, cosmetics, industrial products, biofuels, synthetic oils, animal feed, and fertilizers. However, for these substitutes to become viable, methods for harvesting the cells, including steps of recovering and processing of intracellular products must be efficient and cost-effective in order to be competitive with the refining costs associated with fossil oil derivatives. Current extraction methods used for harvesting microorganisms such as algae to ultimately yield products for use as fossil oil substitutes are laborious and yield low net energy gains, rendering them unviable for today's alternative energy demands. Such previous methods can also produce a significant carbon footprint, exacerbating global warming and other environmental issues. These prior methods, when further scaled up, produce an even greater efficiency loss due to valuable intracellular component degradation and require greater energy or chemical inputs than what is currently financially feasible from a microorganism harvest. For example, the cost per gallon for microorganism bio-fuel is currently approximately nine times the cost of fossil fuel.

All living cells, prokaryotic and eukaryotic, have a plasma transmembrane that encloses their internal contents and serves as a semi-porous barrier to the outside environment. The transmembrane acts as a boundary, holding the cell constituents together, and keeps foreign substances from entering. According to the accepted current theory known as the fluid mosaic model (S. J. Singer and G. Nicolson, 1972, incorporated herein by reference), the plasma membrane is composed of a double layer (bi-layer) of lipids, an oily or waxy substance found in all cells. Most of the lipids in the bilayer can be more precisely described as phospholipids, that is, lipids that feature a phosphate group at one end of each molecule.

Within the phospholipid bilayer of the plasma membrane, many diverse, useful proteins are embedded while other types of mineral proteins simply adhere to the surfaces of the bilayer. Some of these proteins, primarily those that are at least partially exposed on the external side of the membrane, have carbohydrates attached and therefore are referred to as glycoproteins. The positioning of the proteins along the internal plasma membrane is related in part to the organization of the filaments that comprise the cytoskeleton, which helps anchor them in place. This arrangement of proteins also involves the hydrophobic and hydrophilic regions of the cell.

Intracellular extraction methods can vary greatly depending on the type of organism involved, their desired internal component(s), and their purity levels. However, once the cell has been fractured, these useful components are released and typically suspended within a liquid medium which is used to house a living microorganism biomass, making harvesting these useful substances difficult or energy-intensive.

In most current methods of harvesting intracellular products from algae, a dewatering process has to be implemented in order to separate and harvest useful components from a liquid medium or from biomass waste (cellular mass and debris). Current processes are inefficient due to required time frames for liquid evaporation or energy inputs required for drying out a liquid medium or chemical inputs needed for a substance separation. Additionally, such processes are commonly limited to batch processing and are difficult to adapt for continuous processing systems.

Accordingly, there is a need for a simple and efficient procedure for dewatering microorganisms, such as algae, so that they can be harvested and their intracellular products can be recovered and used as competitively-priced substitutes for fossil oils and fossil oil derivatives required for manufacturing of industrial products.

Additionally, the viability of a harvested algae biomass is closely tied to the amount of bacteria or other harmful contaminants that are present within the biomass. For example, contaminants such as bacteria, fungi, rotifers, ciliates, or adverse algae strains can limit the lifetime of the harvesting biomass.

If an algae biomass is contaminated, it is often unsuitable for an intended use and is therefore discarded. Alternately, the algae biomass can be treated with antibiotics, chemicals, or changes in salinity, pH, or other environmental factor to minimize the contamination. Although such treatments have some beneficial effect on extending the life of the algae biomass, the treatments add additional cost, time, and complexity to various processes in which the biomass is used.

### BRIEF SUMMARY

The present invention is generally directed to a system for producing an algae biomass and wastewater that have reduced concentrations of contaminants. The algae and wastewater treated by the system of the present invention can be combined in a heterotrophic growth system in which the growth of the algae is increased due to the reduced concentration of contaminants. The algae grown in this manner also has a longer shelf life due to the lack of contaminants within the harvested algae.

In some embodiments, the present invention is implemented as a method for producing an algae biomass and wastewater having a reduced concentration of contaminants for use in a heterotrophic growth system. A growth medium containing suspended algae is supplied into a first flocculation tank. The first flocculation tank comprises a reactor tube for creating an electric field within the growth medium, the electric field causing the algae to flocculate. The growth medium containing flocculated algae is transferred into a first flotation tank. The first flotation tank comprises a tank containing a plurality of electrodes which cause the formation of gas bubbles which attach to the flocculated algae and lift the flocculated algae to the surface of the growth medium. The floating algae are removed from the surface of the growth media and transferred to a heterotrophic growth system. Wastewater is supplied into a second flocculation tank. The second flocculation tank comprises a second reactor tube for creating an electric field within the wastewater. The second reactor tube includes a cathode and an anode which comprises a titanium ruthenium alloy. When the electric field is created, the anode causes the creation of free chlorine within the fluid leading to the oxidation of the ammonia into nitrite and nitrate. After the ammonia is oxidized, the wastewater is transferred to the heterotrophic growth system such that the wastewater can act as a food for the growth of the algae in the heterotrophic growth system.

In some embodiments, the present invention is implemented as an apparatus for removing ammonia from wastewater. The apparatus comprises a reactor tube for creating an electric field within wastewater containing ammonia. The reactor tube includes a cathode and an anode comprised of a titanium ruthenium alloy. When the electric field is created, the anode causes the creation of free chlorine within the wastewater leading to the oxidation of the ammonia into nitrite and nitrate. The apparatus also comprises a flotation tank connected to the reactor tube. The flotation tank comprises a tank containing a plurality of electrodes which cause the formation of gas bubbles.

In other embodiments, the present invention is implemented as a system for producing an algae biomass and wastewater having a reduced concentration of contaminants for use in a heterotrophic growth system. The system comprises a first apparatus for removing ammonia from wastewater. The first apparatus comprises a first reactor tube for creating an electric field within wastewater containing ammonia. The first reactor tube includes a first cathode and a first anode comprised of a titanium ruthenium alloy. When the electric field is created, the first anode causes the creation of free chlorine within the wastewater leading to the oxidation of the ammonia into nitrite and nitrate. The system includes a second apparatus for harvesting algae using a two-stage process. The second apparatus comprises a second flocculation tank in which the first stage of the two stage process occurs. The second flocculation tank comprises a second reactor tube for creating an electric field within a growth medium containing suspended algae, the electric field causing the algae to flocculate. The second apparatus also includes a second flotation tank in which the second stage of the two stage process occurs. The second flotation tank comprises a second tank containing a plurality of second electrodes which cause the formation of gas bubbles which attach to the flocculated algae and lift the flocculated algae to the surface of the growth medium. The second flotation tank is connected to the second flocculation tank to allow the flocculated algae to flow from the second flocculation tank into the second flotation tank.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates a two-stage algae harvesting apparatus having a first stage flocculation tank and a second stage flotation tank;

FIG. 1B illustrates side views of various possible configurations of electrodes within the second stage flotation tank;

FIG. 1C illustrates a side view of the first stage flocculation tank;

FIG. 2A illustrates the first stage flocculation tank when filled with a growth medium containing suspended algae;

FIG. 2B illustrates the first stage flocculation tank when the algae is flocculated in a batch mode;

FIG. 2C illustrates the first stage flocculation tank when the algae is flocculated in a continuous flow mode;

FIGS. 3A-3D illustrate the process, performed within the second stage flotation tank, of dewatering the flocculated algae using hydrogen bubbles to float the flocculated algae to the surface;

FIG. 4 illustrates an actual implementation of a two-stage algae harvesting apparatus in accordance with one or more embodiments of the present invention;

FIG. 5 illustrates how a two-stage apparatus can be used to produce an algae biomass and treated wastewater having reduced concentrations of contaminants to enhance the growth of the algae during a heterotrophic growth phase; and

FIG. 6 illustrates how the first stage tank of the two-stage apparatus can be used to eliminate contaminants from wastewater.

### DETAILED DESCRIPTION

In this continuation-in-part, a particular use of the algae harvesting methods and apparatuses described in U.S. patent application Ser. No. 13/865,087 (the “Parent Application”) is presented. In the Parent Application, an apparatus having a first stage flocculation tank and a second stage flotation tank is described. This description is reproduced below under the headings: “First Stage Flocculation Tank” and “Second Stage Flotation Tank.” This two-stage harvesting process produces an algae biomass having a reduced concentration of contaminants.

The Parent Application also describes that by using an electrode made of a titanium ruthenium alloy, wastewater can be treated using the two-stage process to remove ammonia and other contaminants from the wastewater. The use of an electrode made of a titanium ruthenium alloy within the two-stage apparatus is described below under the heading: “Additional Features Or Variations.”

It has been found that the algae biomass generated by the two-stage harvesting process can be used in a heterotrophic growth phase during which wastewater treated using the two-stage process can supply the necessary food to the algae to allow the algae to reproduce. Wastewater contains a substantial amount of desirable compounds to encourage algae growth including oxygen, organic carbon, and fertilizers. However, untreated wastewater also contains significant contaminants that are harmful to the algae. Accordingly, for wastewater to be a viable option for use in a heterotrophic growth phase, it must be treated to reduce the contaminant concentration.

The two-stage process can be used to cheaply and efficiently treat the wastewater so that it can be used in the heterotrophic growth phase. Because the algae biomass and the wastewater generated using the two-stage process have reduced concentrations of contaminants than algae biomasses and wastewater treated using other methods, a heterotrophic growth phase can be implemented more quickly and efficiently than in previous solutions.

The specific use of the two-stage method and apparatus to facilitate a heterotrophic growth phase is described below under the heading: “Enhancing A Heterotrophic Growth Phase of Algae Using Treated Wastewater.”

General Discussion of Two-Stage Method and Apparatus for Harvesting Algae

The present invention is generally directed to an apparatus for harvesting algae using a two-stage approach. The two-stage approach includes a flocculation stage and a dewatering stage. The flocculation stage is implemented within a first-stage flocculation tank in which algae suspended within a growth medium is flocculated. The flocculated algae is then fed to a second-stage flotation tank in which electrodes are used to produce hydrogen and oxygen bubbles which attach to the flocculated algae causing the flocculated algae to float to the surface. The mat of floating algae can then be skimmed off the surface of the growth medium.

The algae harvested in this manner are free of harmful substances that are often required in other algae harvesting methods. Additionally, because harmful substances are not used in the two-stage process, the nutrient-rich growth medium can be reused in subsequent algae harvesting.

The apparatus of the present invention can be configured in various sizes. However, in many embodiments, the apparatus can be sized so that it is relatively portable to allow its use in virtually any location. In this way, many entities can employ the apparatus to produce an algae biomass without requiring a large area of land and/or large amounts of electricity as is often required in other harvesting approaches.

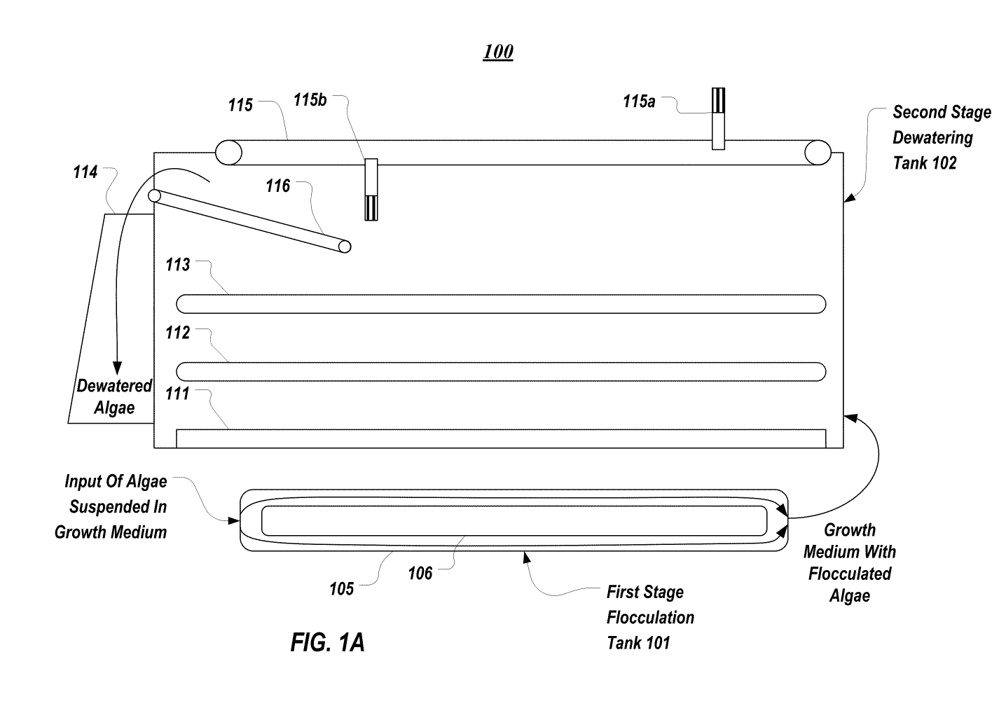

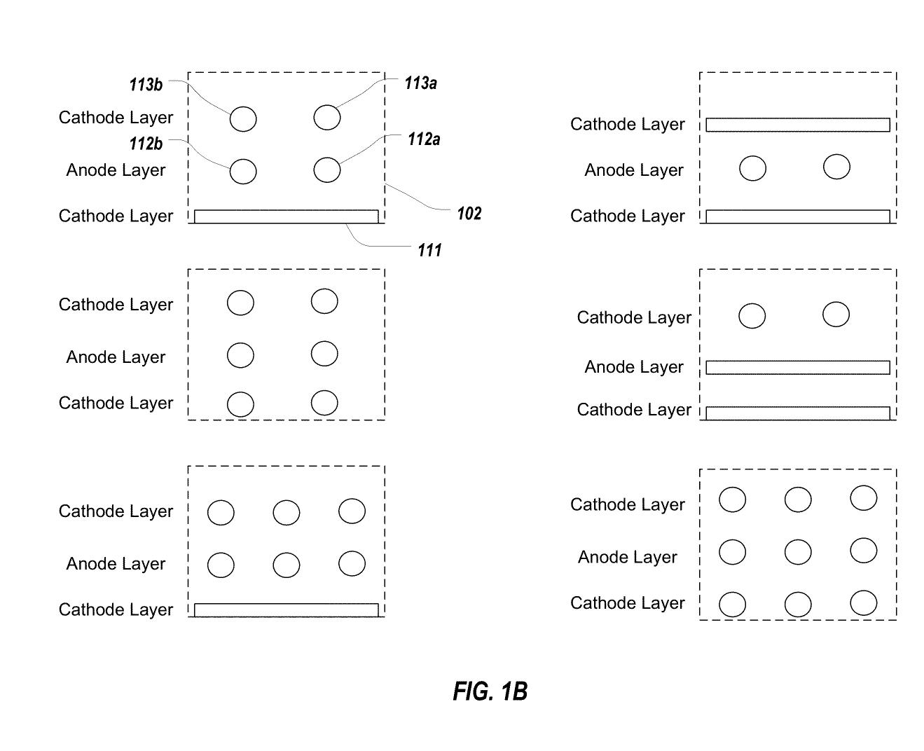

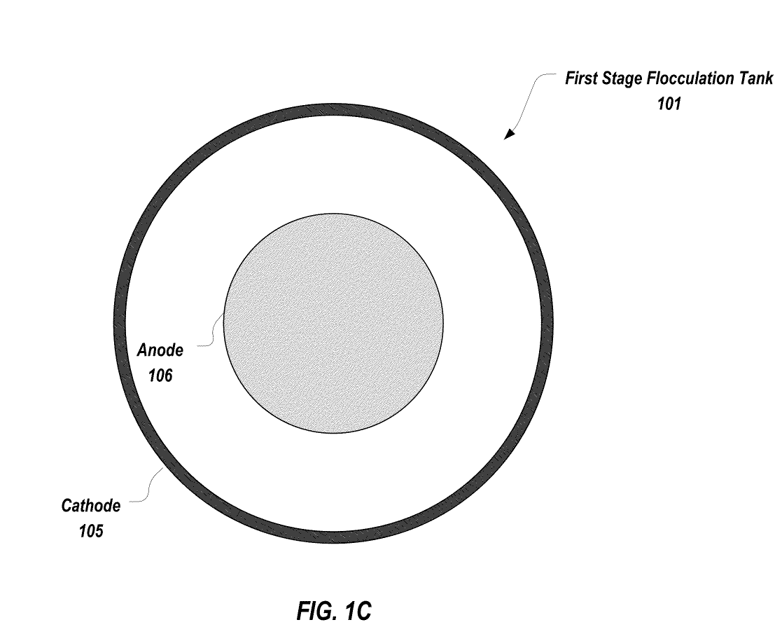

FIG. 1A illustrates an example configuration of an apparatus 100 that harvests algae using the two-stage approach. Apparatus 100 includes two primary components: a first stage flocculation tank 101, and a second stage flotation tank 102.

A growth medium containing suspended algae is input into first stage flocculation tank 101. This growth medium can be obtained in virtually any manner. For example, a dedicated unit for growing algae within water can be connected to first stage flocculation tank 101, or a growth medium otherwise obtained can be directly supplied to first stage flocculation tank 101.

The suspended algae is flocculated (i.e. caused to form clumps) within first stage flocculation tank 101. This flocculation can be caused using an electric current produced by electrodes as will be further described below. Once the algae is flocculated to a desired degree, the growth medium containing the flocculated algae is fed into second stage flotation tank 102.

Second stage flotation tank 102 produces gas (e.g. hydrogen and oxygen) bubbles which rise through the growth medium. While rising, the bubbles attach to the flocculated algae and lift the flocculated algae to the surface. This process results in a mat of algae forming at the surface of the growth medium. Finally, the algae can be collected using conveyors 115 and 116 as will be further described below.

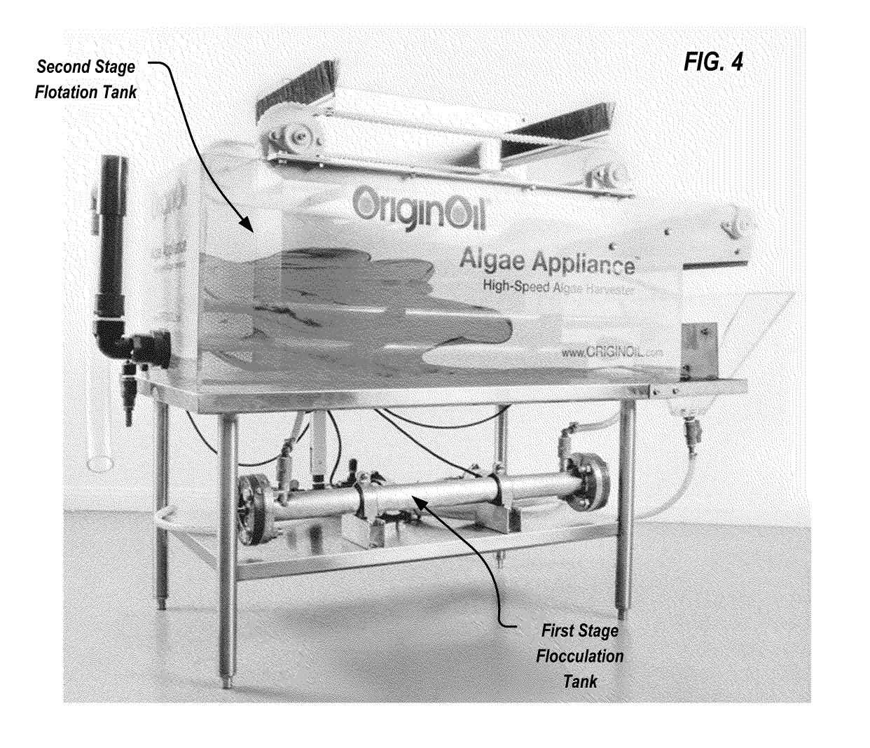

FIG. 4 illustrates an actual implementation of an apparatus in accordance with one or more embodiments of the present invention.

First Stage Flocculation Tank

As shown in FIG. 1A, flocculation tank 101 includes two primary components: a cathode 105 formed by an outer cylinder (e.g. an enclosed pipe or tube), and an anode 106 formed by an inner cylinder (e.g. a pipe or other enclosed cylindrical shape) that is contained within the outer cylinder. Accordingly, the growth medium flows between cathode 105 and anode 106 as shown by the arrows in FIG. 1A. Other shapes other than cylinders can also be used as long as a fluid pathway is formed between the two components. Also, in some embodiments, multiple inner cylinders can be used for anode 106. In some embodiments, the surfaces of cathode 105 and anode 106 which are in contact with the growth medium can include grooves (e.g. rifling) which may decrease the occurrence of build-up on the surfaces.

FIG. 1C illustrates a cross-sectional side view of flocculation tank 101. As shown a space exists between cathode 105 and anode 106 through which the growth medium flows. In some embodiments, this space can be between 0.5 mm and 200 mm wide. A voltage is applied to each of cathode 105 and anode 106 to cause an electric current to pass through the growth medium. This electric current causes the suspended algae in the growth medium to flocculate (i.e. to clump together). In some embodiments, as the algae pass through flocculation tank 101, the cells are exposed to both a magnetic field, causing a cellular alignment, and to an electrical field which induces cellular current absorption. These effects can cause the cells to flocculate.

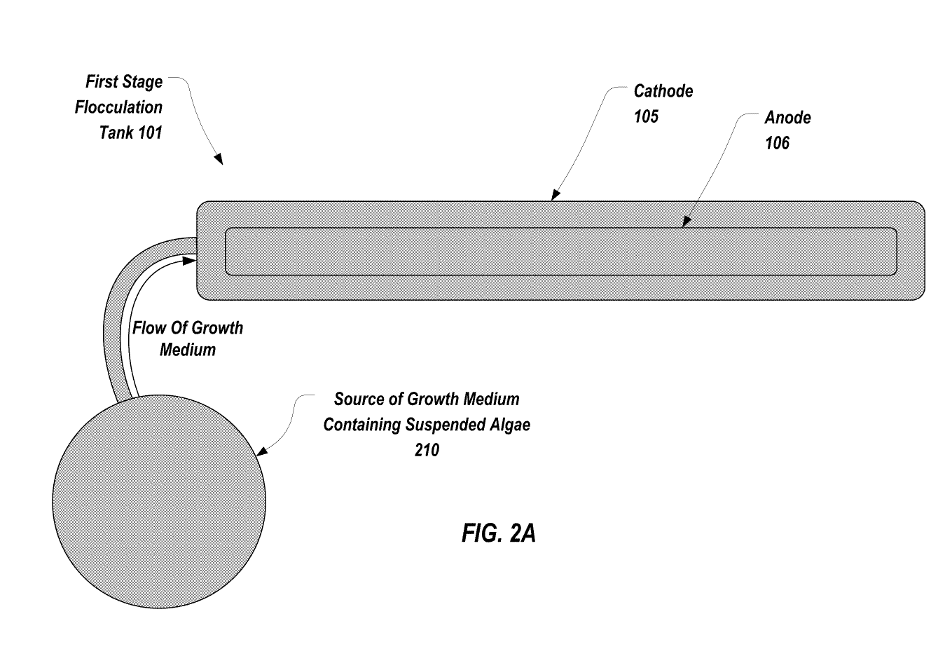

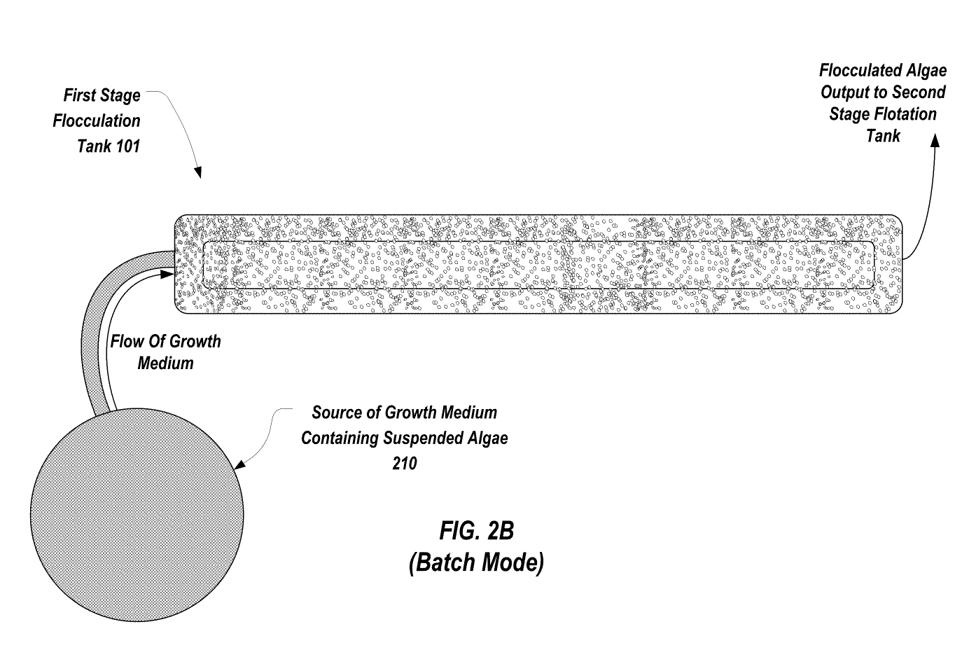

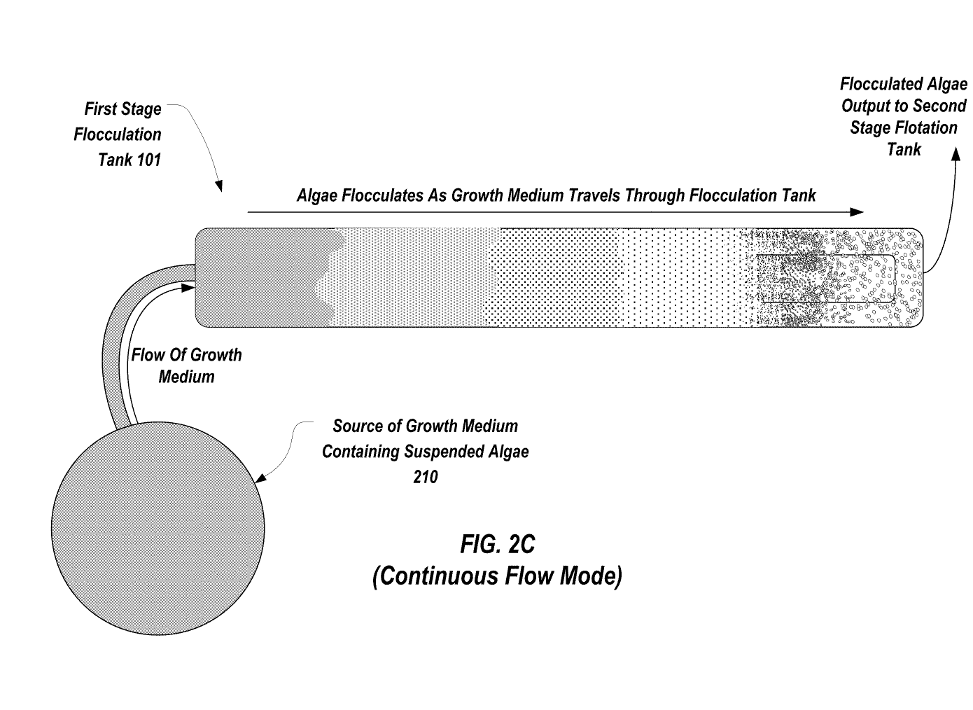

FIGS. 2A-2C illustrate how this flocculation can occur. As shown, a source 210 of growth medium containing suspended algae is connected to flocculation tank 101. Alternatively, growth medium could be supplied manually to flocculation tank 101. The shading in FIG. 2A indicates that the algae are initially suspended in the growth medium.

FIG. 2B illustrates the case where the growth medium is treated in a batch mode. In the batch mode, flocculation tank 101 is initially filled with growth medium containing suspended algae. The growth medium is then subject to the electrical fields generated by cathode 105 and anode 106 until the desired level of flocculation has occurred. In some embodiments, the flocculated algae can be between 1 and 4 mm in size. Then, the growth medium with the flocculated algae is transferred to second stage flotation tank 102. Accordingly, FIG. 2B illustrates that the growth medium within flocculation tank 101 contains clumps of algae which are ready to be transferred to flotation tank 102.

FIG. 2C, in contrast, illustrates the case where the growth medium is treated in a continuous flow mode. In the continuous flow mode, the algae can be flocculated in the same manner as in the batch mode (e.g. by applying an electric current to the growth medium). However, the growth medium can be continuously flowed into flocculation tank at an appropriate rate so that, by the time the growth medium reaches the opposite end of the flocculation tank, the algae has been sufficiently flocculated. This is shown in FIG. 2C with the growth medium at the left end having a similar degree of flocculation as the growth medium in source 210 and the degree of flocculation increasing towards the right end.

Regardless of the mode used to flocculate the algae, flocculation tank 101 can be configured with controls for automatically determining the appropriate settings to ensure that the algae is sufficiently flocculated before exiting flocculation tanks 101. For example, in batch mode, flocculation tank 101 can automatically determine an appropriate duration of time to treat the growth medium or appropriate voltage levels to apply to cathode 105 and anode 106. Similarly, in continuous flow mode, flocculation tank 101 can automatically determine an appropriate flow rate and appropriate voltage levels to apply to cathode 105 and anode 106.

In at least one embodiment, the flow rate through flocculation tank 101 can be 0.1 ml/second per ml of volume. In other embodiments, however, the flow rate is at least 0.5 ml/second per ml of volume or at least 1.0 ml/second per ml of volume. In still other embodiments, the flow rate through the volume is at least 1.5 ml/second per ml of volume. In yet other embodiments, the flow rate through the volume exceeds 1.5 ml/second per ml of volume. In at least one additional embodiment, the flow rate can be controlled by controlling the pressure using a pump or other suitable fluid flow mechanical devices.

In some embodiments, the supplied voltage can be pulsed on and off repeatedly to cause extension and relaxation of the algae cells. According to such embodiments, voltages can be higher and peak amperage lower while average amperage remains relatively low. In such embodiments, this condition or controlled circumstance reduces the energy requirements for operating the apparatus and reduces wear on the anode and cathode pair or pairs. In at least one embodiment, the frequency of the pulses is at least about 500 Hz, 1 kHz, 2 kHz, or kHz. In other embodiments, the frequency is less than 200 kHz, 80 kHz, 50 kHz, 30 kHz, 5 kHz, or 2 kHz. Ranges for the pulse frequency can be any combination of the foregoing maximum and minimum frequencies according to various embodiments.

In some embodiments, an electrical pulse is repeated in frequency to create an electromagnetic field and electrical energy transfer between the electrodes. As this pulsed electrical transfer occurs, an electromagnetic field is produced resulting in the elongation of the algae cells due to their polarity according to certain embodiments. According to further embodiments, the suspended algae absorb electrical input which causes internal cellular components and their liquid mass to swell in size. In such embodiments, and due to swelling, an internal pressure is applied against the transmembrane, however this internal swelling is to be considered as only momentary according to certain embodiments as it is relieved during an off frequency phase of the pulsed electrical input. As mentioned above, in some embodiments, rapid repeating of the on and off electrical frequency rearranges components and creates and/or increases the polar regions in the algae cells. In some embodiments, continuous frequency inputs further produce internal pressures caused by expanded internal component swelling which eventually creates the magnetic/electrostatic attraction causing coagulation/flocculation of the treated cells.

Although this specification primarily describes that the first stage leaves the algae cells intact during the flocculation process, it is also possible to lyse the algae cells during flocculation. For example, by varying the voltage levels/frequency applied to cathode 105 and anode 106 and/or varying the time that the algae cells are subject to the electric current formed between cathode 105 and anode 106, the algae cells can be lysed to thereby release the internal contents of the algae cells. Accordingly, in some embodiments, apparatus 100 can be used to lyse, flocculate, and dewater algae cells.

Second Stage Flotation Tank

Once the algae are flocculated in the growth medium, the growth medium is transferred to flotation tank 102. An electrical field can be applied to the growth medium within flotation tank 102 using electrodes. The electric field increases interface potential between solvent and solute and creates micron-sized bubbles of hydrogen and oxygen gas which lift the flocculated algae to the surface. The algae form a mat at the surface allowing for easy removal of the algae. Also, the mat of algae includes a substantial amount of hydrogen and oxygen gas. The algae can be used with this gas present, or further downstream processes can be performed to recover the gas. For example, the gas can be recovered and used to power apparatus 100 thereby minimizing the energy requirements for using apparatus 100.

Referring again to FIG. 1A, flotation tank 102 includes a cathode plate 111 and a series of stacked anode 112 and cathode 113 rods. FIG. 1B illustrates side views of other configurations of electrodes that can be used within flotation tank 102. For example, at the top left corner of FIG. 1B, the configuration depicted in FIG. 1A is shown. In some embodiments, a plate can be used in place of the rods.

Various other configurations of electrodes can be used. For example, a single cathode and a single anode, two cathodes and a single anode, a single cathode and two anodes, two cathodes and two anodes, or other combinations include one or more cathodes and one or more anodes.

As shown in FIG. 1B, some embodiments provide a two-by-three electrode arrangement, with two vertical columns of three electrodes. The top and bottom rows of electrodes can be cathodes and the middle row can include two anodes. Various other such anode-cathode configurations can be used in embodiments of flotation tank 102. Generally, combinations of between 1 and 20 anodes and between 1 and 20 cathodes can be used depending primarily on the size of flotation tank 102.

Flotation tank 102 also includes conveyor 115 (having rakes 115a and 115b) and conveyor 116 which are used to remove the algae cells from flotation tank 102 and into collector 114 as will be further described below. Other means for removing the algae from the surface of the growth medium can also be used as in known in the art.

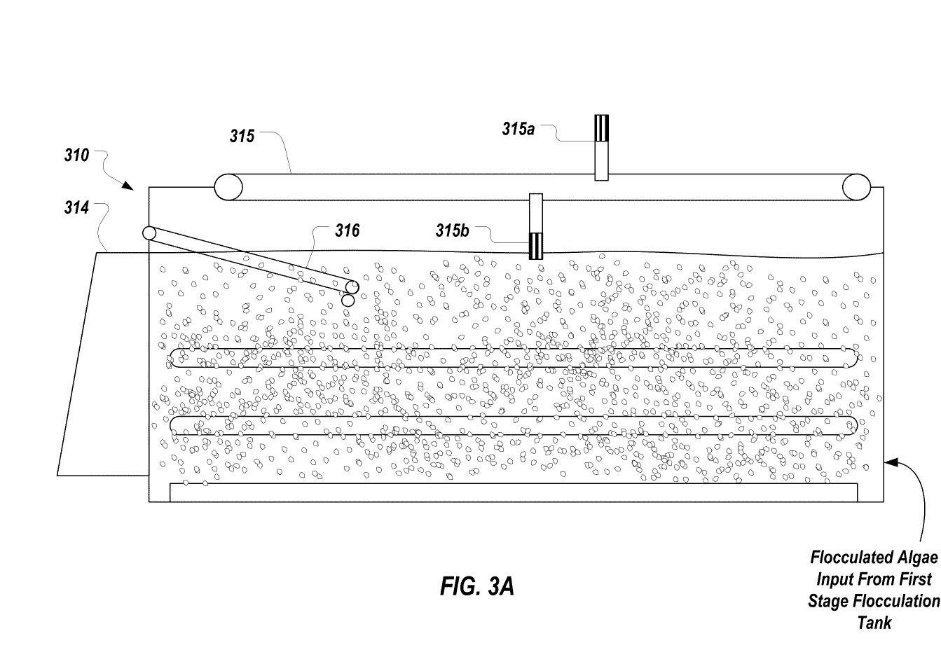

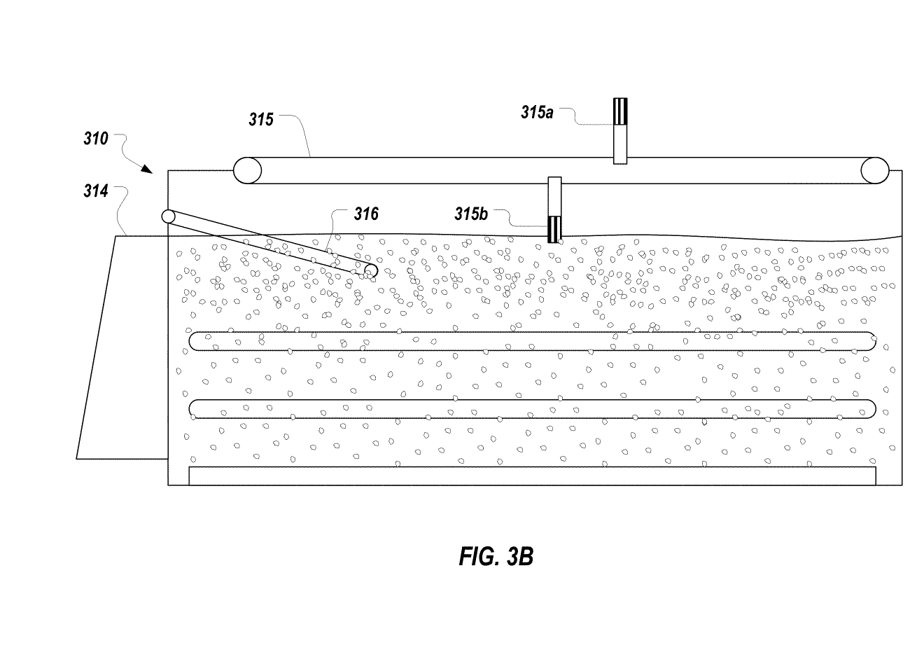

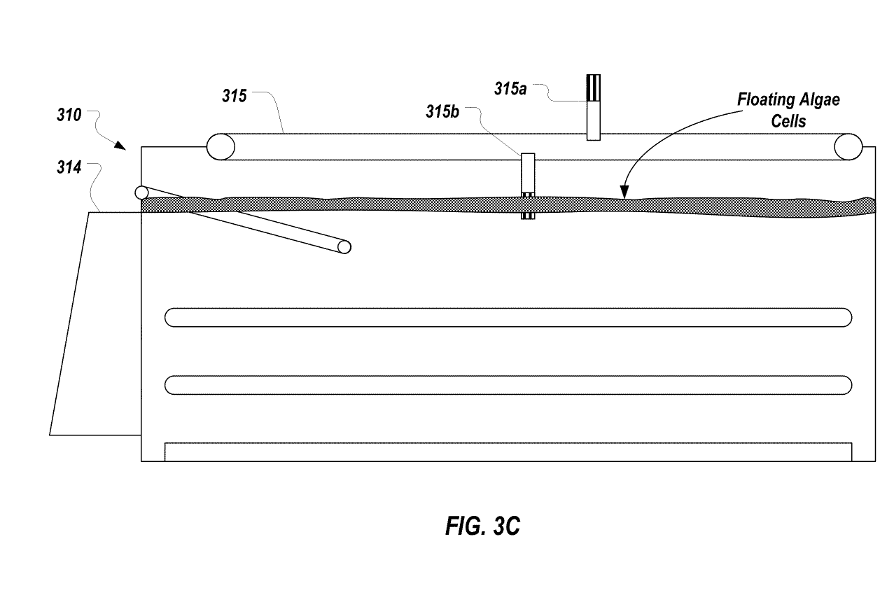

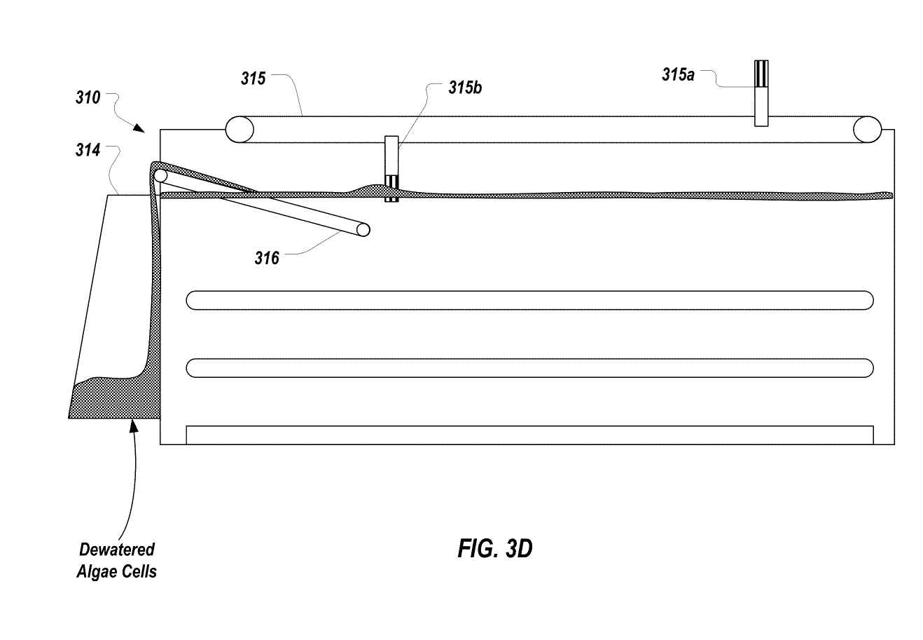

FIGS. 3A-3D illustrate flotation tank 102 to provide an example of how the flocculated algae can be floated to the surface. FIG. 3A illustrates the state of flotation tank 102 when a growth medium containing flocculated algae is passed into flotation tank 102. As stated above, prior approaches for separating algae from the growth medium are difficult, expensive, and oftentimes harmful to the algae making them unsuitable to recover algae that is intended for certain purposes. In contrast, the present invention provides a simple and safe process for recovering the algae cells. This process includes applying an electric field to the growth medium using electrodes 111, 112, and, in some cases, 113.

FIG. 3C illustrates the state of flotation tank 102 after the flocculated algae cells have floated to the surface. FIG. 3C also illustrates that the remaining growth medium underneath the floating clumps is substantially clear to indicate that this process is highly effective at separating the algae from the growth medium. The growth medium, which is nutrient dense, can then be reused.

Finally, FIG. 3D illustrates an example of how the floating algae cells can be removed. As shown, this removal can be performed using rakes 115a, 115b which are rotated over the surface of the growth medium to rake the algae cells towards conveyor 116. Conveyor 116 is rotated to transfer the raked algae cells into collector 114 where it can be retrieved for further processing. Accordingly, this process yields a highly dewatered biomass that can be easily transported and used.

FIGS. 3A-3D generally represent the process as being performed in batches (i.e. the entire growth medium is fully flocculated before any new algae cells are added). However, in some embodiments, this process can be performed on a continuous basis such as by periodically adding new growth media containing flocculated algae.

Gas bubble formation can be facilitated by strategically placing the electrodes in proximity to one another. For example, in some embodiments, the cathode(s) and anode(s) are spaced between about 0.1 inches and about 36 inches apart, between about 0.2 inches and about 24 inches apart, about 0.5 inches and about 12 inches apart, about 0.5 inches and about 6 inches apart, about 3 to about 8 inches apart, about 1 inch to about 3 inches apart, or variations and combinations of these ranges or values within these ranges. The ratio of separation may vary depending on the conductivity of the growth medium and/or the power levels applied to the electrodes. For example, the more saline or conductive the growth medium, the smaller the gap is required for hydrogen and/or oxygen production. In some configurations, the placement of two or more cathodes near a single anode can increase turbulence about the anode, creating a heightened mixing effect that can assist in aggregating and lifting the algae cells.

An operating voltage of between about 1 and about 30 volts, about 1 and about 24 volts, about 2 to about 18 volts, about 2 to about 12 volts, or combinations and intermediate ranges within these ranges, can be applied. For example, a voltage of about 4 volts, 6 volts, 8 volts, 10 volts, 12 volts, 14 volts, 16 volts, 18 volts, 20 volts, 22 volts, 24 volts, 26 volts, 28 volts, 30 volts, and/or combinations of these voltages or ranges encompassing these voltages can be applied. The amperage may vary and generally be between about 1 A to about 20 A, about 2 A to about 15 A, or combinations or intermediate ranges within these ranges. The actual current may reasonably vary depending on the density of the growth medium and its relative conductivity.

In some configurations, it can be desired to provide pulsed power to the electrods. To pulse power, the frequency of pulsing can be varied as can the duty cycle. In this context, the term duty cycle refers to the relative lengths of the on and off portions of each power cycle, and can be expressed, for example, as a ratio of the duration of the on portion of the cycle to the total time for the cycle, or as a ratio of the duration of the on portion of the cycle to the off portion of the cycle, or by stating the on and off durations, or by stating wither the on or off duration and the total cycle duration. Unless otherwise stated or is clear from the context, duty cycle will be stated herein as the ration of on duration to off duration for a cycle.

Accordingly, with embodiments that cycle an electromagnetic field on and off, the duty cycle can be about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.5, about 1:3, about 1:4, about 1:5, about 1:6, about 1:7, about 1:8, about 1:9, or about 1:10. Additionally, the duration of the duty cycle can be varied based upon the flow rate, volume, and/or characteristics of the growth medium.

Additional Features or Variations

The electrodes can be made of a metal, composite, or other material known to impart conductivity, such as, but not limited to silver, copper, gold, aluminum, zinc, nickel, brass, bronze, iron, lead, platinum group metals, steel, stainless steel, carbon allotropes, and/or combinations thereof. Non-limiting examples of conductive carbon allotropes can include graphite, graphene, synthetic graphite, carbon fiber (iron reinforced), nano-carbon structures, and other form of deposited carbon on silicon substrates. In some configurations, the anode and/or the cathode can serve as a sacrificial electrode which is used in the flocculation and/or bubble generation processes. As such, electrodes can include consumable conductive metals, such as iron or aluminum.

In some embodiments, the electrodes (e.g. cathodes 105, 111, 113 and anodes 106, 112) can be comprised of a catalyst-coated metal such as iridium oxide coated titanium. Such metals can enhance the efficiency of the process. For example, by using iridium oxide coated titanium on the anode, the creation of gas bubbles can be facilitated.

Also, in some embodiments, one or more of the electrodes in flotation tank 102 can include numerous perforations or surface textures which allow the growth medium to pass through it. Such perforations and texturing provide an increase in the number of edges on the electrodes, which may facilitate bubble formation. For example, the one or more anodes may be formed as a mesh, grid, or other porous structure. The mesh may include relatively large openings that are larger than a typical clump of algae or sludge particulates in the growth medium. This configuration can advantageously allow for faster flow rates since it allows for greater interfacial contact between the growth medium and the hydrogen generated by the anode. This configuration may be advantageous when a faster flow through is desired or when conductivity of the growth medium is low. Moreover, in some embodiments, growth medium may be introduced into flotation tank 102 at the center of the anode. In this way, the growth medium will flow out one or more holes in the anode and be exposed to gas bubbles.

Although the above described apparatus 100 has shown flotation tank 102 as a separate elevated tank, it is also possible to form the flotation tank as a trench (e.g. in the ground). Using a trench can allow for the processing of greater amounts of growth medium.

In some embodiments, the efficiency of flocculating and/or floating the algae can be increased by adding a protic solvent to the growth medium. For example, the growth medium may be injected with a dilute solution of a protic solvent such as formic acid, n-butanol, isopropanol, n-propanol, ethanol, methanol, and acetic acid, such as of approximately 0.05% by volume. This solution may be mixed into the growth medium at various times. However, in some cases, it is beneficial to add the protic solvent as the electric field of the flocculation process is generated, or just before the batch process occurs.

The above-described apparatus can also be used to remove ammonia from wastewater or other fluids such as in aquaculture environments. To accomplish ammonia removal, the one or more anodes of flocculation tank 101 can be made of a titanium ruthenium alloy. By using a titanium ruthenium alloy, free chlorine is produced in the growth medium when the voltage is applied to the cathode and anode. The free chlorine allows the ammonia to be oxidized eventually resulting in conversion of the ammonia into nitrate, nitrite, and some nitrogen gas.

It has been found that a current density of between 30-50 mA/cm² of the anode is generally preferred to maximize the oxidation of the ammonia into nitrate and nitrite. However, other current densities can also be used, and the ideal density will depend on various characteristics such as the temperature of the wastewater.

Although the removal of ammonia from the wastewater is primarily performed within flocculation tank 101, in such implementations, flotation tank 102 can still be used to remove other undesirable matter from the wastewater such as organic compounds.

Enhancing a Heterotrophic Growth Phase of Algae Using Treated Wastewater

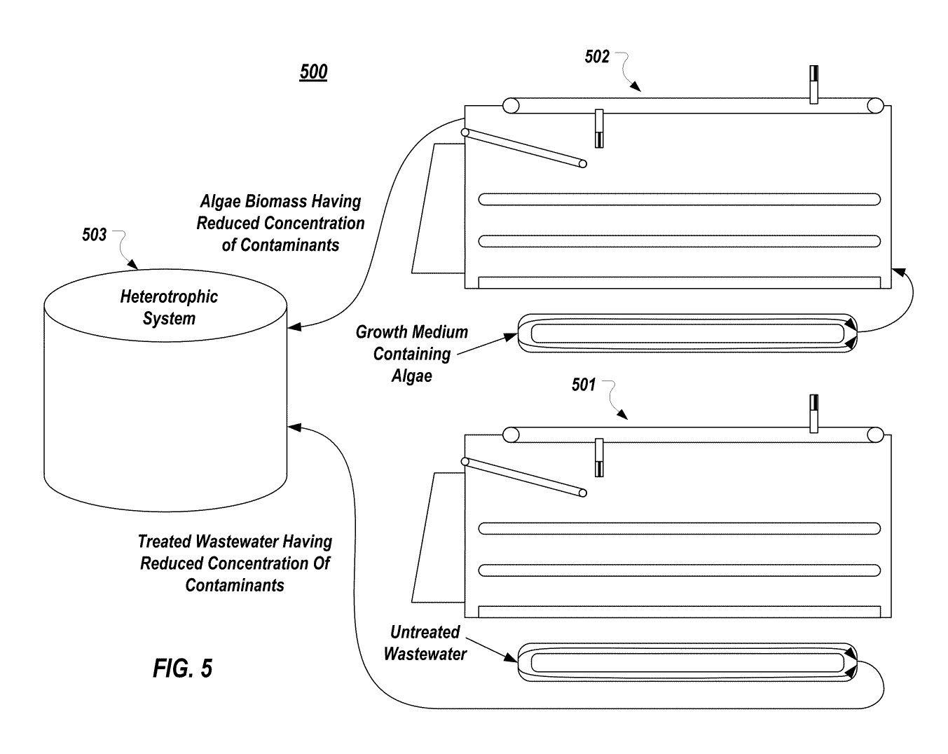

FIG. 5 illustrates a system 500 that produces an algae biomass and treated wastewater having a reduced concentration of contaminants. System 500 is shown as including a first apparatus 501 for treating wastewater and a second apparatus 502 for producing a algae biomass. Both apparatus 501 and apparatus 502 can be structured in the same manner as apparatus 100 described above. In other words, apparatus 502 can be used to produce an algae biomass in the same manner described above.

The two-stage process described above inherently reduces contaminants from a treated substance. For example, because harmful chemicals are not used in the two-stage process, and because of the effect of the flocculation stage, many different types of treated substances will have a reduced concentration of contaminants.

In system 500, apparatus 501 is modified to further enhance the amount of decontamination on wastewater treated in the first stage flocculation tube. This modification is briefly described above as using a titanium ruthenium alloy for one or more of the electrodes. The titanium ruthenium alloy produces free chlorine during operation of the electrodes which, as described above, leads to the oxidation of the ammonia in the wastewater.

In this way, the wastewater can be easily treated to make it suitable for use as a food for the algae biomass during a heterotrophic growth phase. Given the availability of wastewater and its high concentration of desirable components for algae growth (e.g. oxygen, organic carbon, and fertilizers), wastewater is a preferred food source for a heterotrophic system. However, other methods of treated the wastewater to make it suitable for the heterotrophic growth phase have proven unsatisfactory in most cases.

In contrast, the apparatus of the present invention can be used to both generate an algae biomass and to decontaminate wastewater that can then be combined in a heterotrophic system 503. Heterotrophic system 503 can be a dark system which enables algae to be grown continuously (e.g. because no light source is required).

Unlike other approaches, the algae biomass and the wastewater generated using an apparatus configured in accordance with the present invention contain a reduced concentration of contaminants (e.g. bacteria that would compete with the algae, ammonia that would damage the algae, etc.). In other words, the inputs to the heterotrophic system 503 are more pure leading to enhanced growth of the algae. Also, because the algae biomass has less contaminants, it has a longer shelf life (e.g. when used to generate oil or other products).

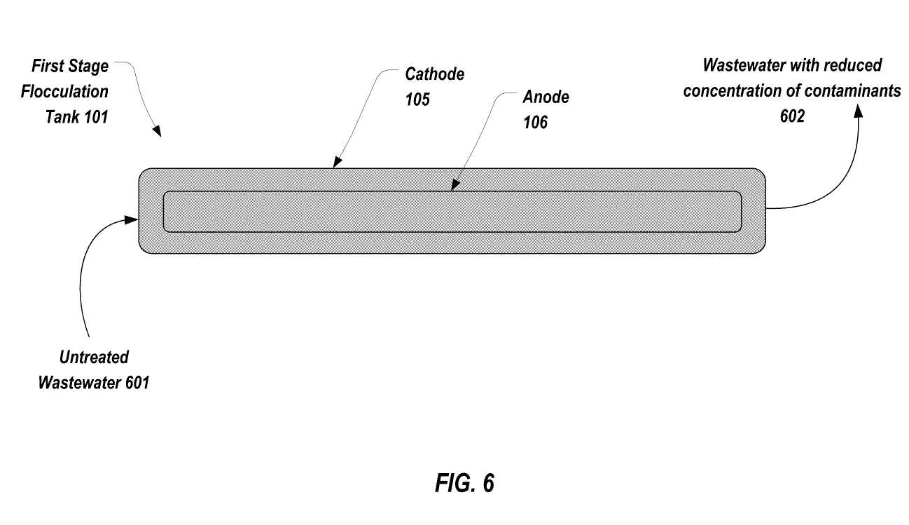

FIG. 6 illustrates first stage flocculation tank 101 when it is used to treat wastewater. Untreated wastewater 601 is input to flocculation tank 101 where it is exposed to the electric field between cathode 105 and anode 106. As stated above, if one of the electrodes, which is typically anode 106, is comprised of a titanium ruthenium alloy, free chlorine is produced which results in the ammonia being oxidized. Also, the electric field can eliminate other contaminants such as bacteria, fungi, etc. from the wastewater. After this process, wastewater 602 with a reduced concentration of contaminants is output.

Wastewater 602 can be used directly after being output from flocculation tank 101, or it can continue into flotation tank 102 where additional matter can be removed from the wastewater if desired. In other words, the treated wastewater can be obtained either after passing through flocculation tank 101 or after passing through flotation tank 102.

To make algae a viable alternative to other fuel sources, it is necessary to increase the fat content of the algae cells. Typically, this has been done using autotrophic growth phases which require an external light source (e.g. the sun). This requirement limits how quickly the algae can grow (i.e. increase their fat content).

Current approaches for growing the algae in a heterotrophic system have required expensive fermenters (such as those used in the beer industry). These fermenters assist in limiting the negative effect of contaminants on the algae cells and in encouraging growth of the desired algae strains. However, because these fermenters are expensive to own and operate, the algae produced using such systems is not a viable alternative to other cheaper fuel sources.

The present invention provides a way to grow algae in a heterotrophic system without the costly requirements of fermenters or such equipment. Because the algae biomass and waster produced by the two-stage apparatus have a reduced concentration of contaminants, the desired algae naturally grow quicker and more efficiently without the need of other equipment or additives. Accordingly, algae with a desired fat content can be produced at a much reduced overall cost.

For example, one or more two-stage apparatuses can be used to produce the necessary ingredients for the heterotrophic growth phase. In some cases, the same apparatus can be used to produce an algae biomass and treated wastewater. The same apparatus can also be used to harvest (including lysing) the algae cells after they have grown to have an adequate fat content. In this way, the requirements for implementing an algae harvesting system are greatly reduced.

In preliminary testing, algae grown by heterotrophic fermentation using the system of the present invention exhibit a much higher cell density and lipid percentage than those grown using autotrophic photosynthesis. Additionally, the growth rate of the algae grown using the system of the present invention is much higher. In a specific study, the lipid percentage of the heterotrophically grown algae have a lipid percentage between 50 and 60 percent, a cell density of greater than 100 grams/L, and a growth rate of greater than 10 grams/L/day. In contrast, autotrophically grown algae exhibited a lipid percentage between 10 and 20 percent, a cell density less than 5 grams/L, and a growth rate of less than 1 gram/L/day.

In a typical implementation, once the heterotrophic algae biomass (e.g. in heterotrophic system 503) has reached a concentration of 3 grams/Liter with 60 percent fat content, the algae can be harvested using the two-stage apparatus as described above. This harvesting can include flocculating and concentrating the algae cells and in some cases lysing and/or hydrogenating the cells. The harvested cells can then be used in many different ways including as a feedstock for a hydropyrolisis refinery.

In some embodiments, the reduced concentration of contaminants that are present in the heterotrophic growth phrase allows the algae cells to reach densities exceeding 150 grams/L. At these densities, the algae cells can be harvested efficiently using a centrifuge. Accordingly, in some embodiments of the present invention, an algae harvesting system can include a centrifuge for harvesting algae from wastewater after a heterotrophic growth phase.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A method for producing an algae biomass and wastewater having a reduced concentration of contaminants for use in a heterotrophic growth system, the method comprising:
supplying a growth medium containing suspended algae into a first flocculation tank, the first flocculation tank comprising a reactor tube for creating an electric field within the growth medium, the electric field causing the algae to flocculate;
transferring the growth medium containing flocculated algae into a first flotation tank, the first flotation tank comprising a tank containing a plurality of electrodes which cause the formation of gas bubbles which attach to the flocculated algae and lift the flocculated algae to the surface of the growth medium;
removing the floating algae from the surface of the growth media and transferring the removed algae to a heterotrophic growth system;
supplying wastewater into a second flocculation tank, the second flocculation tank comprising a second reactor tube for creating an electric field within the wastewater, the second reactor tube including a cathode and an anode, the anode comprising a titanium ruthenium alloy, wherein when the electric field is created, the anode causes the creation of free chlorine within the fluid leading to the oxidation of the ammonia into nitrite and nitrate;
after the ammonia is oxidized, transferring the wastewater to the heterotrophic growth system such that the wastewater can act as a food for the growth of the algae in the heterotrophic growth system.

2. The method of claim 1, wherein the first flocculation tank and the second flocculation tank are the same tank.

3. The method of claim 1, further comprising:
after the algae have grown within the heterotrophic growth system, transferring the wastewater containing the suspended algae into the first flocculation tank to flocculate the algae;
transferring the wastewater containing the flocculated algae into the first flotation tank to cause the flocculated algae to be lifted to the surface of the wastewater; and
removing the floating algae from the surface of the wastewater.

4. The method of claim 3, wherein the wastewater containing the suspended algae is transferred into the first flocculation tank to flocculate the algae after the lipid percentage of the algae cells is greater than 50 percent.

5. The method of claim 3, wherein the wastewater containing the suspended algae is transferred into the first flocculation tank to flocculate the algae after the cell density of the algae cells is greater than 100 grams/L.

6. The method of claim 1, wherein the heterotrophic growth system is a dark system.

7. The method of claim 1, wherein the electric field is created within the second reactor tube by applying a current density of between 30 and 50 mA/cm² of the anode.

8. The method of claim 1, further comprising:
prior to transferring the wastewater to the heterotrophic growth system, transferring the wastewater to a second flotation tank.

9. The method of claim 8, further comprising: removing matter from the wastewater while the wastewater is within the second flotation tank.

10. The method of claim 1, further comprising:
harvesting the algae from the wastewater using a centrifuge.

11. An apparatus for removing ammonia from wastewater, the apparatus comprising:
a reactor tube for creating an electric field within wastewater containing ammonia, the reactor tube including a cathode and an anode, the anode comprising a titanium ruthenium alloy, wherein when the electric field is created, the anode causes the creation of free chlorine within the wastewater leading to the oxidation of the ammonia into nitrite and nitrate; and
a flotation tank, connected to the reactor tube, the flotation tank comprising a tank containing a plurality of electrodes which cause the formation of gas bubbles.

12. The apparatus of claim 11, wherein the electric field is created by applying a current density of between 30 and 50 mA/cm² of the anode.

13. A system for producing an algae biomass and wastewater having a reduced concentration of contaminants for use in a heterotrophic growth system, the system comprising:
a first apparatus for removing ammonia from wastewater, the first apparatus comprising:
a first reactor tube for creating an electric field within wastewater containing ammonia, the first reactor tube including a first cathode and a first anode, the first anode comprising a titanium ruthenium alloy, wherein when the electric field is created, the first anode causes the creation of free chlorine within the wastewater leading to the oxidation of the ammonia into nitrite and nitrate;
a second apparatus for harvesting algae using a two-stage process, the second apparatus comprising:
a second flocculation tank in which the first stage of the two stage process occurs, the second flocculation tank comprising a second reactor tube for creating an electric field within a growth medium containing suspended algae, the electric field causing the algae to flocculate; and
a second flotation tank in which the second stage of the two stage process occurs, the second flotation tank comprising a second tank containing a plurality of second electrodes which cause the formation of gas bubbles which attach to the flocculated algae and lift the flocculated algae to the surface of the growth medium, the second flotation tank being connected to the second flocculation tank to allow the flocculated algae to flow from the second flocculation tank into the second flotation tank.

14. The system of claim 13, wherein the first apparatus further comprises:
a first flotation tank, connected to the first reactor tube, the first flotation tank comprising a first tank containing a plurality of first electrodes which cause the formation of gas bubbles.

15. The system of claim 13, further comprising:
a heterotrophic growth system into which algae removed from the second flotation tank and wastewater removed from the first reactor tube are placed.

16. The system of claim 15, wherein the heterotrophic growth system is a dark system.

17. The system of claim 15, wherein the heterotrophic growth system is fluidly connected to the second apparatus to allow the wastewater and algae within the heterotrophic growth system to be transferred to the second apparatus for harvesting of the algae.

18. The system of claim 17, wherein the heterotrophic growth system is configured to transfer wastewater containing algae to the second apparatus when the algae have a lipid percentage greater than 50 percent.

19. The system of claim 13, further comprising:
a power source that creates the electric field between the first cathode and the first anode, the electric field formed by applying a current density of between 30 and 50 mA/cm² of the anode.

20. The system of claim 13, wherein the second electrodes of the second flotation tank comprise a first cathode layer a second cathode layer and an anode layer positioned between the first and second cathode layers.
